(19) United States
(12) Patent Application Publication
(10) Pub. No.: US 2004/0253327 A1
(43) Pub. Date: Dec. 16, 2004

(54) Compositions and methods for reducing or controlling blood cholesterol, lipoproteins, triglycerides, and sugar and preventing or treating cardiovascular diseases

(76) Inventors: Sarfaraz K. Niazi, Deerfield, IL (US); Anjum Niazi, Deerfield, IL (US)

Correspondence Address: SARFARAZ K. NIAZI, 20 RIVERSIDE DRIVE, DEERFIELD, IL, 60015, US

(21) Appl. No.: 10/250,197

(22) Filed: Jun. 12, 2003

(51) Int. Cl. A61K 35/78; A61K 31/695; A61K 31/555

(52) U.S. Cl. 424/738; 424/757; 424/748; 514/54; 514/63; 514/184

(57) ABSTRACT

This invention provides compositions and methods related to the administration of psyllium husk, Î²-sitosterol, guggul tree extract, guar gum and chromium as a combination to reduce or control blood cholesterol, triglycerides, low density lipoproteins, blood sugar or increasing or controlling high density lipoproteins in a mammal, to reduce arterial plaque build-up, atherosclerosis, in a mammal which may be associated with cardiovascular, cerebrovascular, peripheral vascular, or intestinal vascular disorders.

### BACKGROUND OF INVENTION

This invention relates to compounds and methods that reduce or control levels of cholesterol and triglycerides and glucose, thus preferably inhibiting or arresting the development of atherosclerosis restenosis when administered to mammals, including humans.

The present invention relates generally to compositions and methods for treating hypercholesterolemia, hyperglycemia and atherosclerosis; more particularly, it relates to methods and compositions for treating or preventing atherosclerosis whereby the many and varied problems associated with the disease can be prevented, arrested, substantially alleviated or cured. In the United States and Western Europe, cardiovascular disease and its associated maladies, dysfunctions and complications are a principal cause of disability and the chief cause of death. One specific entity significantly contributing to this pathophysiologic process is atherosclerosis, which has been generally recognized as the leading health care problem both with respect to mortality and health care costs. The American Heart Association estimates that 953,110 persons died of cardiovascular diseases in 1997 (41.2 percent of all deaths), more than the number of mortality for cancer (539,377), accidents (95,644) and HIV (16,516) combined. Furthermore, by association calculations, close to a quarter of the US population suffers from one or more forms of cardiovascular disease. (American Heart Association). The medical costs associated with coronary heart disease are estimated at $95 billion dollars a year. [Gonzalez & Kannewurf, 55 (19) American Journal of Health-System Pharmacy S4-7 (Supp. 1, 1998)]. Diabetes is a major cause of heart and artery diseases compounding the effects of hypercholestrolemia and presence of high triglycerides.

Atherosclerosis is a disease characterized by the deposition of fatty substances, primarily cholesterol, and subsequent fibrosis in the inner layer (intima) of an artery, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages (white blood cells) and smooth muscle fibers. The fibrous plaque of the various forms of advanced atherosclerosis has increased intimal smooth muscle cells surrounded by a connective tissue matrix and variable amounts of intracellular and extracellular lipid. At the luminal surface of the artery, a dense fibrous cap of smooth muscle or connective tissue usually covers this plaque or lesion. Beneath the fibrous cap, the lesions are highly cellular consisting of macrophages, other leukocytes and smooth muscle cells. Deep in this cell-rich region may be areas of cholesterol crystals, necrotic debris and calcification. If allowed to progress, the disease can cause narrowing and obstruction of the lumen of the artery, diminished or occluded blood flow and, consequently, ischemia or infarction of the predominantly affected organ or anatomical part such as the brain, heart, intestine or extremities. The result can be significant loss of function, loss of cellular substance, emergency medical and/or surgical procedures, and significant disability or death. Alternatively, the arterial wall can be severely weakened by the infiltration of the muscular layer with the lipid (cholesterol), inflammatory white blood cells, connective tissue and calcium, resulting in soft and/or brittle areas which can become segmentally dilated (aneurysmal) and rupture or crack leading to organ, limb or even life-threatening hemorrhage. Once the disease has progressed to the stage of significant persistent symptoms and compromised function, the next treatment step has conventionally been artery bypass grafting to repair and/or replace the damaged artery. While coronary artery bypass has become one of the more common major cardiovascular surgical procedures in the United States, surgery clearly is not the solution to the pathologic process. Moreover, there is a significant risk of morbidity and mortality associated with surgery that many patients are reluctant to accept. Indeed, the autogenous veins or arteries used to bypass the disease-impaired arteries undergo atherosclerosis changes postoperatively generally at a faster rate than the original, affected arteries. The Coronary-Artery Surgery Study (CASS) sponsored by the National Heart, Lung and Blood Institute (NHLBI) concluded that certain subsets of patients do not gain any overall statistical benefit from bypass surgery in comparison to other medical treatments. [Carraciolo, 91(9) Circulation 2335-44 (1995).]As an alternative to coronary bypass surgery, certain medications and procedures are used to treat the results of atherosclerosis. These treatments include chelation with ethylene diamine tetra-acetic acid (EDTA) and percutaneous transluminal coronary angioplasty (PTCA). EDTA treatments, however, are still experimental, unproved and potentially as harmful as they are beneficial. PTCA treatments are invasive, of limited application and success and occasionally manifest lethal complications. Highly experimental intra-arterial laser beam plaque vaporization has limited application and requires an open operative approach to affected vessels. It is now well established that vascular blockage and cardiovascular disorders including myocardial infarction, coronary heart disease, hypertension and hypotension, cerebroascular disorders including stroke, cerebral thrombosis and memory loss due to stroke; peripheral vascular disease and intestinal infarction are caused by blockage of arteries and arterioles by atherosclerotic plaque. The production of atherosclerotic plaque formation is multi-factorial in its production.

Hypercholeterolemia, especially elevated levels of low-density lipoprotein cholesterol (LDL) is an important risk factor for atherosclerosis and arteriosclerosis and associated diseases. Lipoproteins are spherical particles with the non-polar triglycerides and cholesteryl esters A in the hydrophobic core, the polar lipids, phospholipids and free cholesterol on the surface with apolipoproteins. When the amount of cholesterol entering the body increases, the pools of sterol within liver cells expands and the receptors that clear LDL from the blood down-regulate, thus increasing LDL levels in the blood. When cholesterol intake is constant, some long-chain saturated fatty acids further suppress the hepatic LDL receptor whereas several unsaturated fatty acids have the opposite effect. Lipoprotein (a) [Lp(a)] has emerged as a plasma lipoprotein linked to both diseases of the coronary arteries, the carotid and the cerebral arteries. It is structurally related to LDL and possesses one molecule of apolipoprotein B₁₀₀ per particle. Macrophages express the scavenger receptor that readily recognizes oxidatively modified Lp (a). [Marcovina & Morrisett, Current Opinions in Lipidology 136-145 (1995).] Cholesterol levels below 200 mg/dl are considered “desirable.” A Scandinavian study showed that reduction of cholesterol reduced mortality associated with coronary artery disease (CAD) by 42% over six year period and reduced overall mortality by 30%. [Goodman & Gilman's the Pharmacological Basis of Therapeutics (J. Hardman & L. Lipman, 9^{th} Ed. 1996)] [Hereinafter “J. Hardman”].

Researchers have shown that a 1 mMol/L increase in triglyceride levels produces a 76% increase in cardiovascular disease risk in women and a 31% increase in men. Austin, 83 (9B) American Journal of Cardiology 13F-16F (1999). Even in patients with established disease, lowering of LDL cholesterol to between 2 and 2.5 mmol/L retards its progression and may even lead to regression. [Illingsworth, 41(20) Drugs 151-160 (1991).] It is recommended that persons with elevated cholesterol concentrations above 240 mg/dL (6.2 mM/L) receive treatment and that those with borderline values between 200-239 mg/dL (5.2 to 6.2 mM/L) be further evaluated according to the presence of risk factors for coronary artery disease including the sex of the patient, post-menopausal status, a low plasma concentration of HDL cholesterol (below 35 mg/dL [0.9 mM/L]), positive family history, smoking, hypertension and diabetes mellitus. [Expert Panel on Detection, Evaluation, and Treatment of High Blood Cholesterol in Adults, 269(23) J. Am. Medical A. 3015-3023 (1993).] Other factors include obesity, hypertriglyceridemia, sedentary lifestyle, steroid use, Î²-adrenergic blocking agents, some diuretics and genetic factors. [Frohlich & Pritchard, 22 Clinical Biochemistry 417-433 (1989).] By the 1980's, it was recognized that HDL levels could be more important in predicting atherosclerotic disease than LDL and that HDL may prevent the development of coronary artery disease. Factors such as smoking, obesity, hypertriglyceridemia, genetic factors and lack of exercise are major causes of reduced serum HDL. HDL cholesterol lipoproteins move excess cholesterol from the extrahepatic organs to the liver for excretion. [Dietschy, 65 Am. J. Clinical Nutrition 1581S-9S (1997).] There is evidence that virtually every body tissue is capable of at least some cholesterol synthesis from the precursor acetyl-coenzyme A (CoA). HDL continuously carries back to the liver an amount of cholesterol equal to the amount synthesized and taken up as LDL by all extrahepatic organs except endocrine glands. There is a second LDL transport process that is receptor independent. Removal of free cholesterol from arterial wall cells may be an important mechanism by which HDL plays an anti-atherogenic role.

The earliest recognized gross lesion in atherogenesis is the fatty streak, characterized by an accumulation of cells loaded with cholesteryl esters (“foam cells”) just beneath the vascular endothelium. The LDL receptor in the arteries gives rise to foam cells and fatty streaks, the earliest lesion in atherosclerosis, but there is also a receptor-independent mechanism for their formation. This has been demonstrated by the development of lesions rich in macrophage-derived foam cells, even in patients and animals deficient in LDL receptors, and the failure to produce foam cells from normal monocytes and monocyte derived macrophages incubated with LDL. This led researchers to explore the possibility of a post-secretory modification of LDL before it is taken up into foam cells by a new, specific receptor: the “scavenger receptor.” [Steinberg, 320(14) New Eng. J. Medicine 915-924 (1989).] Common medications used to lower plasma cholesterol levels include clofibrate, dextrothyroxine sodium, colestipol hydrochloride, gemfibrozil, probucol, niacin/nicotinic acid and cholestyramine resin. These drugs and their associated treatments, however, generally are directed only at the cause, and not the result, of atherosclerosis and have not been shown to be effective in reversing the plaque deposition and degenerative changes in the arterial walls. These pharmacological agents also have many other shortcomings such as, for example, adverse side effects (hypertension, cardiac arrhythmias, gastrointestinal disturbances, headache, hypersensitivity, etc.), contraindications (heart, liver or kidney disease, pregnancy, etc.), requirement for lifelong conscientious administration, difficulty in maintaining consistent patient compliance, variable reliability and high cost.

Other therapies have been used to lower cholesterol levels. These include: dietary changes, Bruce, 19(1) Journal of the American College of Nutrition 61-7 (2000); fiber and psyllium, Knopp, 17(1) American Journal of Preventive Medicine 18-23 (1999), Burton & Mannien, 668 Medica Scandinavica 9104 (Supp. 1982); Vitamins C, E, and carotenoids, Anonymous 20(1) European Heart Journal 725-41 (1999), Azen, 94(10) Circulation 2369-72 (1996), Hodis, 273(23) J. Am. Medical A 1849-54 (1995), Kothari 28(1) Acta Biologica Academiae Scientiarum Hungaricae 111-4 (1977); L-carnitine, Stefanutti, 149(2) Clinica Terapeutice 115-9 (1998), Elisaf, 18(5) Am. J. Nephrology 416-21 (1998); fatty acids, Leng, 4(4) Vascular Medicine 219-26 (1999); fatty acids eicosapentanoic acid (fish oil) and garlic, Morcos, 89(10) J. Nat. Medical A.-673-8 (1997);.beta glucan, U.S. Pat. No. 6,020,324 to Jamas, et. al.; and, amino acids, U.S. Pat. No. 5,248,688 to Dudrick.

Non-insulin dependent diabetes mellitus (type 2 diabetes) is a metabolic disorder characterized by hyperglycemia, which occurs due to insulin deficiency, insulin resistance and reduced glucose tolerance. In people with diabetes mellitus, blood sugar levels are too high. These high levels occur because glucose remains in the blood rather than entering cells, where it belongs. But for glucose to pass into a cell, insulin must be present, and the cell must be “hungry” for glucose. People with type 1 diabetes do not make insulin. For them, insulin are the only way to keep blood sugar levels down. People with type 2 diabetes mellitus tend to have two problems. They do not make quite enough insulin, and the cells of their bodies do not seem to take in glucose as eagerly as they should. The first treatment for type 2 diabetes mellitus is often meal planning for blood glucose (sugar) control, weight loss, and exercising. But sometimes these measures are not enough to bring blood sugar down near the normal range. The next step is taking a medicine that lowers blood sugar levels. There are two kinds of medicines: oral medications and insulin injections. Approximately 50 percent of the 17 million Americans who have been diagnosed with type 2 diabetes mellitus are currently required to take a combination of oral medications, along with their recommended diet and exercise program, in order to control their blood sugar.

Careful control of blood sugar reduces the long-term effects of type 2 diabetes on the body's circulatory system. We have learned a great deal about this from three recent studies (the Diabetes Control and Complications Trial [DCCT: Diabetes Control and Complications Trial Research Group: The effect of intensive treatment of diabetes on the development and progression of long-term complications in insulin-dependent diabetes mellitus. N Engl J Med 329:977986, 1993] study and the U.K. Prospective Diabetes Study [UKPDS] UK Prospective Diabetes Study Group: UK Prospective Diabetes Study Group: Tight blood pressure control and risk of macrovascular and microvascular complications in type 2 diabetes (UKPDS 38). BMJ 317:703713, 1998) and Stockholm Diabetes Intervention Study: Reichard P, Nilsson B Y, Rosenqvist V: The effect of long-term intensified insulin treatment on the development of microvascular complications of diabetes mellitus. N Eng J Med 329:304309, 1993. As a result of these important large-scale research studies, the ADA has set new therapeutic goals. These include a target level of 7% for HbA1c (glycated hemoglobina measure of blood glucose control that provides information about average glucose levels over months rather than minutes or hours, which is all the information that blood glucose levels can provide); 80-120 mg/dl (4.4-6.6 mMol/l) for fasting plasma glucose (FPG); and 100-180 mg/dl (5.5-10 mMol/l) for postprandial (after eating a meal) glucose.

All diabetes mellitus oral medications sold today in the US are members of several classes of drugs based on their mechanism of action. Drugs that stimulate insulin secretion include sulfonylureas and benzoic acid derivatives, drugs that suppress hepatic glucose production include biguanides, drugs that sensitize body to insulin include thiazolidinediones, drugs that reduce postprandial plasma glucose include alpha-glucosidase inhibitors.

The first class of drugs stimulates the beta cells to release more insulin. This class is represented by sulfonylurea drugs that have been in use since the 1950s. This class of agents was the mainstay of the treatment of type 2 diabetes for many years. They stimulate the body to produce, or secrete, more insulin. Other beneficial effects of the sulfonylurea class of drugs include suppression of glucose production in the liver and enhancement of the body's ability to dispose of excess glucose into fat and muscle tissue. Sulfonylureas remain the most popular group of medications today. In part, this may be a function of physicians' habits but an important factor is their low cost. Many types of sulfonylureas are now generic and are among the cheapest medications available for the treatment of diabetes. A drawback of sulfonylureas is that, on average, they lose effectiveness for 44% of patients within six years of beginning their use. Individual patients may have a more prolonged course of successful sulfonylurea treatment and others may need supplements even earlier than six years. Failure occurs more rapidly in younger, more hyperglycemic individuals and in those with lower insulin secretion at the start of treatment. While this may sound like a high rate of failure, it is important to remember that some of the newer drugs have not been in use for very long and we do not yet know how effective they are long-term. Hypoglycemia and weight gain are the two most frequent side effects of these drugs. An early study, the UGDP (University Group Diabetes Program), published in the 1970s, and also raised the possibility that sulfonylureas might make heart disease worse. However, the UKPDS study, mentioned above, found that sulfonylureas are no more likely to increase coronary artery disease than any of the other agents tested (insulin and metformin). Sulfonylureas, however, have little or no effect on blood lipid concentrations. Chlorpropamide is the only first-generation sulfonylurea still in use today. Other first generation sulfonylureas include tolbutamide and tolazamide; some of the most popular brands of these first generation sulfonylureas include Orinase^{Â}® and Diabinese^{Â}®. The second-generation sulfonylureas are used in smaller doses than the first-generation drugs. There are three second-generation drugs: glipizide, glyburide, and glimepiride (Diabeta^{Â}®, Micronase^{Â}®, Glynase^{Â}®, Glucotrol^{Â}®, Glucotrol XL^{Â}®, Amaryl^{Â}®). These drugs are generally taken one to two times a day, before meals. All sulfonylurea drugs have similar effects on blood sugar levels, but they differ in side effects, how often they are taken, and interactions with other drugs.

The second class of drugs also stimulates insulin secretion like sulfonylureas but are not sulfonylureas and work by a different mechanism as well; these are benzoic acid derivatives or meglitinide such as repaglinide (Prandin^{Â}®). It is rapidly absorbed and quickly metabolized in the body requiring three doses each day. For some patients, this presents an inconvenience but for others, with erratic lifestyles that lead them, occasionally, to miss meals, skipping repaglinide at the same time appears to be very effective. Repaglinide seems to have little effect on lipids and can, like the sulfonylureas, cause weight gain and hypoglycemia.

The third class of diabetes mellitus drugs sensitizes the body to the insulin that is already present. This class is represented by metformin (Glucophage^{Â}®), which is a biguanide. It lowers blood sugar by helping insulin work well, mostly in the liver. It is usually taken two times a day. A side effect of metformin may be diarrhea, but this is improved when the drug is taken with food. Metformin is not a new medication, although it was only approved by the FDA for use in the United States in 1995. Its primary effect is to inhibit the liver's production of glucose and, possibly, to stimulate the process of transporting glucose into muscle, a process which requires insulin. Thus it only works when there is insulin around, for example in type 2 diabetes, but not type 1 diabetes, which is characterized by insulin deficiency. Exactly how metformin works is not well understood. Metformin can be used as a first line of therapy. It is useful for patients who are obese because it does not cause the weight gain seen with sulfonylureas; it may even bring about some degree of weight loss. Metformin is also as capable as the sulfonylureas in reducing HbA1c. An additional benefit of metformin is its positive effect upon lipid metabolismit reduces blood triglyceride and LDL (the “bad”) cholesterol levels by about 10% and also lowers fatty acids. Side effects can be a problem with metformin. Up to 30% of patients develop gastrointestinal complaints, though these may be mild and temporary, especially if dosages are brought up slowly. The largest concern with metformin is the potential to produce a build up of lactic acid. However, this is a very rare side effect of the drug, particularly if care is taken not to prescribe metformin when it is contraindicated. Contraindications for this drug include evidence of kidney disease, significant liver disease, chronic alcoholism or congestive heart failure. Hypoglycemia and weight gain, are not on the listed side effects of metformin.

The fourth class of drugs enhances insulin action in muscle, fat and other tissues and are known as insulin sensitizers such as thiazolidinediones (TZDs), comprised of group of glitazones, rosiglitazone, and pioglitazone. They require the presence of insulin in order to work, so TZDs are not indicated for type 1 (“insulin dependent diabetes”) and certain other varieties of diabetes. TZDs are effective in reducing HbA1c. They are also effective in combination with either sulfonylureas or metformin. Compared to other drugs, it takes a patient a long time to see the benefits of the TZDs. For this reason, doses should not be increased until after 4-6 weeks, the time it normally takes for maximal biological effect to occur. About 25% of patients do not respond to TZDs. Some TZDs also have beneficial effects on blood lipids. Troglitazone has a lipid lowering effect and increases HDL, or high-density lipoprotein (“the good cholesterol”). Pioglitazone also decreases triglycerides. The major side effect, seen with troglitazone, the first TZD to be approved by the FDA, is liver damage. The effects observed range from an elevation in liver enzymes, which is reversible, to liver failure, which has caused death in a small number of patients. Because of this dangerous side-effect, the FDA, in March 2000, A® removed troglitazone (Rezulin^{Â}®) from the market. Two other members of the TZD class that have recently been approved by the FDA, A® A® rosiglitazone and pioglitazone (Avandia^{Â}®, Actos^{Â}®), do not appear to cause liver damage. However, the FDA requires regular monitoring of liver enzyme levels with these drugs as well. Other side effects of TZD are mild elevations of LDL (the “bad”) cholesterol and fluid retention TZDs do not cause hypoglycemia when used alone.

The fifth class of oral drugs slows or blocks the breakdown of starches and certain sugarsglucosidase inhibitors. After you eat, the food is digested, and then passes into the bloodstream and, thus, the level of sugar in the blood rises. Glucosidase inhibitors act in the intestine to block the action of enzymes that are responsible for breaking down complex carbohydrates into simple sugars. This delayed breakdown of carbohydrates helps slow down their absorption into the bloodstream and, thus, slow down the increase in blood glucose levels after a meal. These medicines are not usually used for primary therapy unless a patient appears to have large increases in blood glucose after meals (“postprandial”). Glucosidase inhibitors are most useful in combination with other drugs. Gastrointestinal side effects are common, affecting up to 30% of patients. Bloating, flatulence, diarrhea and abdominal discomfort and pain are the major complaints. However, these side effects can be reduced by eating less carbohydrates in the diet. Hypoglycemia is not often seen but, if the patient develops low blood glucose levels, he/she must be treated with glucose, not complex carbohydrates. This is because the action of these drugs, which prevent breakdown of complex carbohydrates in the intestine, will be unable to rapidly correct blood glucose concentrations. Weight gain does not occur with these drugs. This class is represented by: and miglitol (Precose^{Â}®Glyset^{Â}®). Miglitol can interfere with the bioavailability of ranitidine and propranalol.

Because the drugs listed above act in different ways to lower blood sugar, they may be used together in combinations. For example, a biguanide and a sulfonylurea may be used together. Many combinations can be used. Combinations of glibenclamide and metformin are patented under U.S. Pat. No. 6,303,146 to Bonhomme, et al., and RE 37,330 (U.S. Pat. No. 5,922,769) to Barelli, et al., for example. The use of sulfonylureas and biguanides in monotherapy, in most cases, allows obtaining an effective glycometabolic control for some years, if an appropriate diet and behavioral regimen are kept. Nevertheless, the efficacy of the therapy with oral hypoglycemic agents can decrease with time. After positive starting responses, which can last 4-5 years, monotherapy becomes ineffective in a considerable percentage of patients. These are the so-called “secondary failures” of the therapy with oral hypoglycemic agents. Such a failure is estimated to occur each year in 5-10% of the patients under therapy with sulfonylureas, therefore after 10 years, only 50% of the patients still show a satisfactory response. Since sulfonylureas are capable of stimulating insulin release, but are not capable of acting on insulin resistance, and biguanides are able to act on insulin resistance, whereas they are not able to stimulate insulin secretion, the therapeutic rationale of said studies suggested the use of combined formulations of medicaments capable of finding a remedy for both the deficiency in insulin secretion and the insulin-resistance condition. The combined therapy (sulfonylurea+biguanide) plays therefore a specifically important therapeutic role, since it allows obtaining an effective metabolic control in those patients with diabetes mellitus of type 2, in which the therapy with only sulfonylureas or only biguanides becomes ineffective with time. Two biguanides are also used in the oral therapy of diabetes mellitus of type 2: phenformin and metformin. Although the former is still widely used, a number of data in literature clearly show that metformin exerts an effective normoglycemic action with no risk of lactic acidosis in the patients, as it can occur in some cases when using phenformin. Therefore, it is generally accepted that metformin is the preferred biguanide in the therapy of diabetes mellitus of type 2. Recently, a combination of glyburide and metformin has been introduced for patients who may fail to respond to sulfonylureas.

The growing population of both type I and type II diabetics around the world has continued to stimulate research in the field of diabetes control through measures to control blood sugar levels. Many pharmaceutical companies are working to produce new and novel classes of drugs as well as new insulin sensitizers and new stimulators of insulin secretion. One example, which is expected to be available shortly, is nateglinide (Starlix^{Â}®), a rapid-onset, short-duration drug that is similar to repaglinide in effect, though quite different chemically. This drug is an effective stimulator of insulin secretion is used prior to meals and may be prove to be useful as primary therapy. Whereas combinations of various groups of antidiabetic or hypoglycemic agents are common, whether used concurrently or in a single dosage form, combinations of these agents with botanical products has never been studied or proposed. Given the nature of diabetes treatment, which involves a lifetime of drug administration, it is appropriate to investigate safe combinations and also the use of safer natural products.

The lethal combination of hypercholesterolemia and hyperglycemia is now well acknowledged; it is now established that glycosylated hemoglobin is a significant factor in the formation of atheroma. There is therefore a need to reduce both, the blood sugar levels (in postprandial situation) for blood cholesterol, even in those people who are not diagnosed with diabetes. The combination of ingredients and the resultant composition described below produces surprising synergistic effects. The invention presented here provides a combination that provides benefits much higher than what could be predicted from the sum of each ingredient. In addition, the need for a combination therapy is fully met by the proposed invention.

Guggul Extract The use of guggulipids in lowering blood cholesterol has also been described in the literature. Guggul is the yellowish resin (or gum) that is produced by the mukul Commiphora mukul tree, a small, thorny plant that grows throughout northern India. Guggul is also referred as guggul gum, guggal, gugglesterone, guggul, gugulu and gum gugal. Guggul plays a major role in the traditional herbal medicine of India. It is often combined with other herbs and used in the treatment of arthritis, skin diseases, pains in the nervous system, obesity, digestive problems, infections in the mouth, and menstrual problems. The mukul myrrh tree is closely related to the Commiphora Mukul tree (or common myrrh). Myrrh was one of the first medicines with hieroglyphic notation of use during ancient Egyptian times depicting its many uses. With such a close relation, many scientists believe that Guggul may have many of the same properties as Myrrh as even their ancient status is similar. The mukul myrrh (Commiphora mukul) tree is a small, thorny plant distributed throughout India. Guggul and gum guggulu are the names given to a yellowish resin produced by the stem of the plant. This resin has been used historically and is also the source of modern extracts of guggul.

Indian researchers discovered an ancient Sanskrit medical text, Sushruta Samhita, in the 1960s. This classical medical text prescribed guggul for the treatment of medoroga, a disease that closely resembles the symptoms of high cholesterol and hardening of the arteries. Indian scientists subsequently tested animals and found that guggul gum both lowered cholesterol levels and protected against the development of hardening of the arteries. These trials culminated in a pilot study that examined guggul's effectiveness in humans. The Indian government has approced guggul as a treatment for high cholesterol.

Studies show that a 14-27% of LDL cholesterol and 22-30% of triglycerides levels were reduced when guggul was given to men and women with high cholesterol for 12 weeks with no change in diet or exercise. Several clinical studies were published in the Indian Journal of Medicine (volume 84) in 1986, Indian Pharmacoepia and in the Journal of the Association of Physicians in India (vol. 34 & 37) all stating the efficacy of guggul in lowering LDL cholesterol and triglycerides. Dr. David Moore and his team at the Baylor College of Medicine in Houston found that the guggulsterone, the active ingredient in the Guggul extract, blocks the activity of a receptor in the liver's cells called Farnesoid X Receptor (FXR). Later, Dr. David Mangelsdorf at University of Texas Southwestern Medical Center in Dallas confirmed that the guggul blocked the receptor and affected how cholesterol is metabolized. Two compounds, Z-guggulsterone and E-guggulsterone, appear to be responsible for guggul lipid's cholesterol-lowering effects. Guggul also appears to boost levels of “good” cholesterol although the exact mechanism is unknown. Both of these actions help prevent Atherosclerosis. Guggul is also an antioxidant, which helps stop the oxidization of cholesterol and the subsequent hardening of the arteries. Given below is a survey of literature on guggul effects on human body: Guggulsterone is an FXR antagonist in coactivator association assays but acts to enhance transcription of bile salt export pump. J Biol Chem 2003 Jan. 13. Cui J, Huang L, Zhao A, Lew J L, Yu J, Sahoo S, Meinke P T, Royo 1, Pelaez F, Wright S D.

The hypolipidemic natural product guggulsterone acts as an antagonist of the bile acid receptor. Mol Endocrinol 2002 July;16(7):1590-7 Wu J, Xia C, Meier J, Li S, Hu X, Lala D S.

A natural product that lowers cholesterol as an antagonist ligand for FXR. Science 2002 May 31;296(5573):1703-6. Urizar N L, Liverman A B, Dodds D T, Silva F V, Ordentlich P, Yan Y, Gonzalez F J, Heyman R A, Mangelsdorf D J, Moore D D.

A safe, effective, herbal antischistosomal therapy derived from myrrh. Am J Trop Med Hyg 2001 December;65(6):700-4 Sheir Z, Nasr M, Massoud A, Salama 0, Badra G A, El-Shennawy H, Hassan N, Hammad S M.

Furanosesquiterpenoids of Commiphora myrrha. J Nat Prod 2001 November;64(11):1460-2. Zhu N, Kikuzaki H, Sheng S, Sang S, Rafi M M, Wang M, Nakatani N, DiPaola R S, Rosen R T, Ho C T.

Simultaneous determination of E- and Z-guggulsterones in dietary supplements containing Commiphora mukul extract (guggulipid) by liquid chromatography.

J AOAC Int2001 January-February;84(1):24-8. Nagarajan M, Waszkuc T W, Sun J.

Alternative therapies: Part II. Congestive heart failure and hypercholesterolemia. Am Fam Physician 2000 Sep. 15;62(6):1325-30 Morelli V, Zoorob R J. Gugulu (Commiphora mukul) induces triiodothyronine production: possible involvement of lipid peroxidation. Life Sci 1999;65(12):PL 137-41. Panda S, Kar A.

Hypolipidemic and antioxidant effects of Commiphora mukul as an adjunct to dietary therapy in patients with hypercholesterolemia. Cardiovasc Drugs Ther 1994 August;8(4):659-64. Singh R B, Niaz M A, Ghosh S.

Recent nutritional approaches to the prevention and therapy of cardiovascular disease. Prog Cardiovasc Nurs 1997 Summer;12(3):3-2. Kendler B S.

Conceptual and methodologic challenges of assessing the short-term efficacy of Guggulu in obesity: data emergent from a naturalistic clinical trial. J Postgrad Med 1995 January-March;41(1):5-7 Bhatt A D, Dalal D G, Shah S J, Joshi B A, Gajjar M N, Vaidya R A, Vaidya A B, Antarkar D S.

Beneficial effects of Allium sativum, Allium cepa and Commiphora mukul on experimental hyperlipidemia and atherosclerosis—a comparative evaluation. J Postgrad Med 1991 July;37(3):132-5. Lata S, Saxena K K, Bhasin V, Saxena R S, Kumar A, Srivastava V K.

Clinical trials with gugulipid. A new hypolipidaemic agent. J Assoc Physicians India 1989 May;37(5):323-8. Nityanand S, Srivastava J S, Asthana O P.

Hypolipidemic activity of guggal resin (Commiphora mukul) and garlic (Alium sativum linn.) in dogs (Canis familiaris) and monkeys (Presbytis entellus entellus Dufresne). Biochem Exp Biol 1980;16(4):421-4. Dixit V P, Joshi S, Sinha R, Bharvava S K, Varma M.

Satyavati G V. Gum guggul (Commiphora mukul) The success of an ancient insight leading to a modern discovery. Indian J Med 1988;87:32735.

Nityanand S, Kapoor N K. Hypocholesterolemic effect of Commiphora mukul resin (Guggal). Indian J Exp Biol 1971;9:36777.

Singh K, Chander R, Kapoor N K. Guggulsterone, a potent hypolipidaemic, prevents oxidation of low density lipoprotein. Phytother Res 1997;11:2914.

Mester L, Mester M, Nityanand S. Inhibition of platelet aggregation by guggulu steroids. Planta Med 1979;37:3679.

Malhotra S C, Ahuja M M S, Sundarum K R. Long-term clinical studies on the hypolipidemic effect of Commiphora mukul (guggul) and clofibrate. Ind J Med Res 1977;65:3905.

Nityanand S, Srivastava J S, Asthana O P. Clinical trials with gugulipida new hypolipidemic agent. J Assoc Phys India 1989;37:3238.

Antonio J, Colker C M, Torina G C, et al. Effects of a standardized guggulsterone phosphate supplement on body composition in overweight adults: A pilot study. Curr Ther Res 1999;60:2207.

Thappa D M, Dogra J. Nodulocystic acne: oral gugulipid versus tetracycline. J Dermatol 1994;21:72931.

Brown D, Austin S. Hyperlipidemia and Prevention of Coronary Artery Disease. Seattle, Wash.: NPRC, 1997, 46.

Guar Gum Guar gum (E412, also called guaran) is extracted from the seed of the leguminous shrub Cyamopsis tetragonoloba, where it acts as a food and water store. Guar gum is a galactomannan similar to locust bean gum consisting of a (1â□□4)-linked Î²-D-mannopyranose backbone with branchpoints from their 6-positions linked to Î±-D-galactose (i.e. 1â□□6-linked-Î±-D-galactopyranose). There are between 1.5-2 mannose residues for every galactose residue. Guar gum is made up of non-ionic polydisperse rod-shaped polymers consisting of molecules (longer than found in locust bean gum) made up of about 10,000 residues. Higher galactose substitution also increases the stiffness (i.e. decreases the flexibility) but reduces the overall extensibility and radius of gyration of the isolated chains. The galactose residues prevent strong chain interactions as few unsubstituted clear areas have the minimum number (about 6) required for the formation of junction zones. Of the different possible galactose substitution patterns, the extremes of block substitution and alternating substitution give rise to the stiffer, with greater radius of gyration, and most flexible conformations respectively (random substitution being intermediate). If the galactose residues were perfectly randomized, it unlikely that molecules would have more than one such area capable of acting as a junction zone, so disallowing gel formation. A block substitution pattern, for which there is some experimental evidence, would allow junction zone formation if the blocks were of sufficient length. Enzymatic hydrolysis of some of the galactose side chains (e.g. using legume Î±-galactosidase) may allow guar gum to be used to replace a dwindling locust bean gum supply.

Guar gum is an economical thickener and stabilizer. It hydrates fairly rapidly in cold water to give highly viscous pseudoplastic solutions of generally greater low-shear viscosity when compared with other hydrocolloids and much greater than that of locust bean gum. High concentrations (˜1%) are very thixotropic but lower concentrations (˜0.3%) are far less so. Guar gum is more soluble than locust bean gum and a better emulsifier as it has more galactose branch points. Unlike locust bean gum, it does not form gels but does show good stability to freeze-thaw cycles. Guar gum shows high low-shear viscosity but is strongly shear-thinning. Being non-ionic, it is not affected by ionic strength or pH but will degrade at pH extremes at temperature (e.g. pH 3 at 50 Â° C). It shows viscosity synergy with xanthan gum. With casein, it becomes slightly thixotropic forming a biphasic system containing casein micelles. Guar gum retards ice crystal growth non-specifically by slowing mass transfer across solid/liquid interface.

Many leguminous plant seeds contain Galactomannans. Guar Gum is obtained from the seeds of Cyamopsis tetragonolobus, an annual leguminous plant originating from India and Pakistan. It is also cultivated in the United States. Guar fruit is a pod; its seeds have an average diameter of about 5 mm.

Controlled clinical trials have demonstrated the beneficial effects of guar gum. Open studies in hypercholesterolaemia with guar gum, a dietary fiber obtained from Cyamopsis tetragonolobus, have shown 10% to 15% reductions in serum concentration of total cholesterol and 10% to 20% in serum concentration of low density lipoproteincholesterol after short term treatment. [Long term effects of guar gum on lipid metabolism after carotid endarterectomy. Juha-Pekka Salenius, et al., BMJ 1995;310:95-96 (14 Chromium Chromium is also indexed as Glucose Tolerance Factor (GTF Chromium). Chromium is an essential trace mineral that helps the body maintain normal blood sugar levels. In addition to its well-studied effects in diabetes, preliminary research has found that chromium supplementation also improves glucose tolerance in people with Turner's syndromea disease linked with glucose intolerance. Chromium may also play a role in increasing HDL (“good”) cholesterol while lowering total cholesterol levels. Chromium, in a form called chromium picolinate, has been studied for its potential role in altering body composition. Preliminary research in animals and humans suggested that chromium picolinate increases fat loss and promotes a gain in lean muscle tissue. Double-blind research has also reported a reduction in body fat and body weight in people given 400 mcg of chromium (as chromium picolinate) per day for three months. However, other studies have failed to show a significant effect of chromium picolinate on body composition. The best source of chromium is true brewer's yeast. Nutritional yeast and torula yeast do not contain significant amounts of chromium and are not suitable substitutes for brewer's yeast. Chromium is also found in grains and cereals, though much of it is lost when these foods are refined. Some brands of beer contain significant amounts of chromium.

Most people eat less than the U.S. National Academy of Science's recommended range of 50200 mcg per day. The high incidence of adult-onset diabetes suggests to some doctors that many people should be supplementing with small amounts of chromium. In supplemental amounts (typically 50300 mcg per day), chromium has not been found to cause toxicity in humans. While there are a few reports of people developing medical problems while taking chromium, a cause-effect relationship was not proven. One study suggested that chromium in very high concentrations in a test tube could cause chromosomal mutations in ovarian cells of hamsters. Chromium picolinate can be altered by antioxidants or hydrogen peroxide in the body to a form that could itself create free radical damage. In theory, these changes could increase the risk of cancer, but so far, chromium intake has not been linked to increased incidence of cancer in humans.

Chromium supplementation may enhance the effects of drugs for diabetes (e.g., insulin, blood sugar-lowering agents) and possibly lead to hypoglycemia. Therefore, people with diabetes taking these medications should supplement with chromium only under the supervision of a doctor.

Saner G, YÃ¼zbasiyan V, Neyzi O, et al. Alterations of chromium metabolism and effect of chromium supplementation in Turner's syndrome patients. Am J Clin Nutr 1983;38:5748.

Riales R, Albrink M J. Effect of chromium chloride supplementation on glucose tolerance and serum lipids including high-density lipoprotein of adult men. Am J Clin Nutr 1981;34:26708.

Wang M M, Fox E Z, Stoecker B J, et al. Serum cholesterol of adults supplemented with brewer's yeast or chromium chloride. Nutr Res 1989;9:98998.

Page T G, Southern L L, Ward T L, et al. Effect of chromium picolinate on growth and serum and carcass traits of growing-finishing pigs. J Anim Sci 1993;71:65662.

Lefavi R, Anderson R, Keith R, et al. Efficacy of chromium supplementation in athletes: emphasis on anabolism. Int J Sport Nutr 1992;2:11122.

McCarty M F. The case for supplemental chromium and a survey of clinical studies with chromium picolinate. J Appl Nutr 1991;43:5966.

Kaats G R, Blum K, Fisher J A, Adelman J A. Effects of chromium picolinate supplementation on body composition: a randomized, double-masked, placebo-controlled study. Curr Ther Res 1996;57:74756.

Kaats G R, Blum K, Pullin D, et al. A randomized, double-masked, placebo-controlled study of the effects of chromium picolinate supplementation on body composition: a replication and extension of a previous study. Curr Ther Res 1998;59:37988.

Hallmark M A, Reynolds T H, DeSouza C A, et al. Effects of chromium and resistive training on muscle strength and body composition. Med Sci Spt Ex 1996;28:13944.

Sterns D M, Belbruno J J, Wetterhahn K E. A prediction of chromium (Ill) accumulation in humans from chromium dietary supplements. FASEB J 1995;9:16507.

Sterns D M, Wise J P, Patierno S R, Wetterhahn K E. Chromium (III) picolinate produces chromosome damage in Chinese hamster ovary cells. FASEB J 1995;9:16439.

Speetjens J K, Collins R A, Vincent J B, Woski S A. The nutritional supplement chromium (III) tris(picolinate) cleaves DNA. Chem Res Toxicol 1999; 12:4837.

Garland M, Morris J S, Colditz G A, et al. Toenail trace element levels and breast cancer. Am J Epidemiol 1996;144:65360.

Cerulli J, Grabe D W, Guathier I, et al. Chromium picolinate toxicity. Ann Pharmacother 1998;32:42831.

Shannon M. Alternative medicines toxicology: a review of selected agents. J Clin Toxicol 1999;37:70913.

Wasser W G, Feldman N S. Chronic renal failure after ingestion of over-the-counter chromium picolinate. Ann Intern Med 1997;126:410 [letter].

Cerulli J, Grabe D W, Gauthier I, et al. Chromium picolinate toxicity. Ann Pharmacother 1998;32:42831.

Martin W R, Fuller R E. Suspected chromium picolinate-induced rhabdomyolysis. Pharmacotherapy 1998;18:8602.

Offenbacher E G. Promotion of chromium absorption by ascorbic acid. Trace Elements Electrolytes 1994;11:17881.

Psyllium Psyllium, flea seed, or Plantago seed is native to South Asia. The seeds are used. In addition to its traditional and current use for constipation, psyllium was also used topically to treat skin irritations, including poison ivy reactions and insect bites and stings. It has also been used in traditional herbal systems of China and India to treat diarrhea, hemorrhoids, bladder problems, and high blood pressure. Psyllium is a bulk-forming laxative and is high in both fiber and mucilage. Psyllium husk has a long history of use in traditional and herbal medicine and has been in use in the United States over 60 years ago. Psyllium husk is derived from the seed or leaves of the Plantago ovata plant. Besides Plantago ovata, psyllium is also known as Ispaghula and Ispagol. Plantago ovata is an annual herb native to Asia, the Mediterranean region, and North Africa. Psyllium grows in sand and silty soils. Currently, psyllium is extensively cultivated in India and Pakistan. India provides about 85% of the psyllium available in the world market. The US is the world's largest importer of psyllium husk. Psyllium has a long history of use throughout the world and has been used in traditional medicine in the United States, Europe, India, and China. Psyllium is a bulk forming fiber. Other fibers that belong to the class of bulk forming fibers are cellulose, methylcellulose, sodium carboxymethylcellulose, karaya, malt soup extract, polycarbophil, and wheat bran. Bulk forming fibers are laxatives because of their water holding properties. They exert their action primarily through mechanical effects by bulking the colonic contents and shortening transit time.

Currently in the United States, psyllium husk is most often used as a bulk fiber laxative, in foods or in various fiber supplements. In 1998, the Food and Drug Administration (FDA) authorized the use of health claim in the labeling of foods and dietary supplements containing psyllium husk. The health claim may state that diets low in saturated fat and cholesterol that include 7 grams of soluble fiber per day from psyllium may reduce the risk of heart disease by lowering cholesterol levels in the blood. The US FDA authorizes health claims on food labels under provisions of the Nutrition Labeling and Education Act of 1990 to ensure that claims are accurate and not misleading to consumers. Health claims are authorized only if, based on the totality of publicly available scientific evidence, there is significant scientific agreement that the claim is true. The psyllium health claim is an amendment to the FDA regulation published on Jan. 23, 1997, which allows a health claim on the association between soluble fiber from whole oats and a reduced risk of coronary heart disease. In allowing the whole oats-coronary heart disease health claim, FDA acknowledged the likelihood that soluble fiber from sources other than whole oats could affect blood lipid levels and thus lower the risk of heart disease. However, since soluble dietary fibers are a family of very heterogeneous substances that vary greatly in their effect on risk of coronary heart disease, a case-by-case approach is necessary to evaluate petitions for health claims for soluble fiber. The new amendment adds psyllium to the sources of soluble fiber allowed to carry the claim for the nutrient-disease relationship of soluble fiber and coronary heart disease. During this rulemaking, FDA evaluated placebo-controlled studies that tested an intake of 10.2 grams of psyllium husk (about 7 grams of soluble fiber) per day as part of a diet low in saturated fat and cholesterol. These studies showed consistently significant blood total- and LDL-cholesterol lowering effects. Foods carrying the health claim must provide at least 1.7 g of soluble fiber from psyllium husk per reference amount customarily consumed of the product. This single-serving size, multiplied by 4 eating occasions per day, totals the 7-gram per day intake of the controlled studies. The soluble fiber of psyllium comes from the dried husk of the psyllium seed. Because some foods containing psyllium husk can be difficult to swallow, foods carrying the psyllium health claim must also have a label statement advising of the need to consume the food with adequate amounts of liquid and to avoid eating the food if one has difficulty swallowing.

Psyllium has been a subject of clinical investigation for its various beneficial properties. The laxative properties of psyllium are due to the swelling of the husk when it comes in contact with water. This forms a gelatinous mass that keeps feces hydrated and soft. The resulting bulk stimulates a reflex contraction of the walls of the bowel, followed by emptying. (Leung A Y, Foster S. Encyclopedia of Common Natural Ingredients Used in Food, Drugs, and Cosmetics, 2d Ed. New York: John Wiley & Sons, 1996, 4279). One observational study found that psyllium seeds successfully treated constipation due only to poor lifestyle (low fiber, low exercise, etc), not when an actual disease was the cause. (Voderholzer W A, Schatke W, MÃ¼hldorfer B E, et al. Clinical response to dietary fiber treatment of chronic constipation. Am J Gastroenterol 1997; 92: 958).

Numerous double-blind studies confirm psyllium can lower total cholesterol and low-density lipoprotein (LDL), the “bad” cholesterol (Anderson J W, Allgood L D, Turner J, et al. Effects of psyllium on glucose and serum lipid response in men with type 2 diabetes mellitus and hypercholesterolemia. Am J Clin Nutr 1999; 70: 46673). However, levels of high-density lipoprotein (HDL) cholesterol (the “good” cholesterol) are not affected. (Oson B H, Anderson S M, Becker M P, et al. Psyllium-enriched cereals lower blood total cholesterol and LDL cholesterol, but not HDL cholesterol, in hypercholesterolemic adults: Results of a meta-analysis. J Nutr 1997; 127: 1973-80). The cholesterol-lowering effect of psyllium has been reported in children, (Davidson M H, Dugan L D, Burns J H, et al. A psyllium-enriched cereal for the treatment of hypercholesterolemia in children: A controlled, double blind, crossover study. Am J Clin Nutr 1996; 63(1): 96-102) as well as in adults. Recent studies report that patients with mild to moderately elevated cholesterol levels can achieve a sustained reduction of about 10% in cholesterol levels by consuming psyllium twice a day and adhering to the American Heart Association's (AHA) Step 1 Diet. (Jenkins D J, Kendall C W, Vuksan V, Vidgen E, Parker T, Faulkner D, Mehling C C, Garsetti M, Testolin G, Cunnane S C, Ryan M A, Corey P N., Soluble fiber intake at a dose approved by the US Food and Drug Administration for a claim of health benefits: serum lipid risk factors for cardiovascular disease assessed in a randomized controlled crossover trial. Am J Clin Nutr. 2002 May;75(5):834-9).

In an open study, people with ulcerative colitis remained in remission just as long when they took 20 grams of ground psyllium seeds BID with water as when they took the medication mesalamine. (Fernandez-Banares F, Hinojosa J, Sanchez-Lombrana J L, et al. Randomized clinical trial of Plantago ovata seeds (dietary fiber) as compared with mesalamine in maintaining remission in ulcerative colitis. Am J Gastroenterol 1999; 94: 427-33). The combination of the two was slightly more effective than either alone.

Psyllium is known to alter the hydrophobicity of bile acids and lowers fecal lithocholic and isolithocholic acids. Psyllium inhibits cholesterol stone formation by reducing biliary cholesterol saturation index. Psyllium thus provides a protective action by selectively decreasing biliary cholesterol and CDCA. Psyllium increases fecal bile acid secretion and thus acts to reduce blood lipids. The high fiber in psyllium purports to reduce gall stone disease. There is reduced bacterial conversion of the primary bile acids to secondary bile acids or metabolites in the presence of psyllium. (Chaplin M F, Chaudhury S, Dettmar P W, Sykes J, Shaw A D, Davies G J. Effect of ispaghula husk on the fecal output of bile acids in healthy volunteers. J Steroid Biochem Mol Biol 2000 April; 72(5): 283-92).

The role of soluble fiber in the prevention of cancer, particularly gastrointestinal carcinoma is well established. However, the data emerging out of clinical trials are inconclusive regarding the potentiating role of psyllium in preventing cancer of the gastrointestinal tract. (Murphy J, Stacey D, Crook J, Thompson B, Panetta D. Testing control of radiation-induced diarrhea with a psyllium bulking agent: a pilot study. Can Oncol Nurs J 2000 Summer;10(3):96-100).

Psyllium has shown significant effect on reducing appetite primarily by producing a feeling of fullness and also by reducing the absorption of fats in the food. Psyllium does not act by slowing down the gastric emptying of hydrosoluble nutrients, but by increase in the time allowed for intestinal absorption, as suggested by the flattening of the postprandial serum glucose, insulin and triglycerides curves. (Turnbull W H, Thomas H G. The effect of a Plantago ovata seed containing preparation on appetite variables, nutrient and energy intake. Int J Obes Relat Metab Disord 1995 May; 19(5): 338-42).

Psyllium is known to inhibit dietary pancreatic lipase enzyme. Glucose formation from starch catalyzed by amylase is inhibited in the presence of bran, guar gum and psyllium, suggesting that the observed effect is also of functional significance. (Hansen W E. Effect of dietary fiber on pancreatic lipase activity in vitro. Pancreas 1987; 2(2): 195-8).

One of the most recent observations about the potential use of psyllium is in preventing rectal leakage of oil in subject taking the new class of lipase enzyme inhibitors such as orlistat (U.S. Pat. No. 6,251,421 to Niazi). Many gastrointestinal side effects resulting from excess unabsorbed oil in the rectal area are obviated by a suitable dosing of psyllium. Psyllium delays fermentation of high amylose diets. Contrary to popular belief, psyllium does not increase gaseous production in the intestine. There is insignificant degradation of psyllium by the intestinal bacteria. (Zaman V, Manzoor S M, Zaki M, Aziz N, Gilani A U. The presence of antiamoebic constituents in psyllium husk. Phytother Res 2002 February;16 (1):78-9).

Psyllium diet attenuates the salt-accelerated hypertension in rats by a possible mechanism of increased fecal excretion of sodium absorbed into the psyllium. The insulin related systolic pressure rise is muted by psyllium diets. Psyllium increases blood glucose disposal by increasing skeletal muscle plasma membrane GLUT-4 content without PI3-kinase activation. (Frape D L, Jones A M. Chronic and postprandial responses of plasma insulin, glucose and lipids in volunteers given dietary fiber supplements. Br J Nutr 1995 May; 73(5): 733-51).

Literature describes a variety of other applications of psyllium such as use in the healing of surface wounds, as a demulcent, antacid, etc. (Westerhof W, Das P K, Middelkoop E, Verschoor J, Storey L, Regnier C. Mucopolysaccharides from psyllium involved in wound healing. Drugs Exp Clin Res 2001;27(5-6):165-75).

Probably due to its soluble-fiber content, psyllium has also improved glucose tolerance in some people with diabetes mellitus. (Florholmen J, Arvidsson-Lenner R, Jorde R, Burhol P G. The effect of Metamucil on postprandial blood glucose and plasma gastric inhibitory peptide in insulin-dependent diabetics. Acta Med Scand 1982; 212: 237-95.; Foster S. Herbs for Your Health. Loveland, Colo.: Interweave Press, 1996, 745; Rodriguez-Moran M, Guerrero-Romero F, Lazcano-Burciaga G. Lipid- and glucose-lowering efficacy of plantago psyllium in type II diabetes mellitus. J Diabetes Complications 1998; 12: 27-38; Anderson J W, Allgood L D, Turner J, et al. Effects of psyllium on glucose and serum lipid response in men with type 2 diabetes mellitus and hypercholesterolemia. Am J Clin Nutr 1999; 70: 466-73). A large number of other studies have confirmed the beneficial effects of psyllium on the glycemic index of foods as well as the control of postprandial sugar levels. (Sierra M, Garcia J J, Fernandez N, Diez M J, Calle A P. Therapeutic effects of psyllium in type 2 diabetic patients. Eur J Clin Nutr 2002 September;56(9):830-42.; Sierra M, Garcia J J, Fernandez N, Diez M J, Calle A P, Sahagun A M. Effects of ispaghula husk and guar gum on postprandial glucose and insulin concentrations in healthy subjects. Eur J Clin Nutr 2001 April;55(4):235-43; Anderson J W, Allgood L D, Turner J, Oeltgen P R, Daggy B P. Effects of psyllium on glucose and serum lipid responses in men with type 2 diabetes and hypercholesterolemia. Am J Clin Nutr 1999 October; 70(4): 466-73; Frati Munari A C, Benitez Pinto W, Raul Ariza Andraca C, Casarrubias M. Lowering glycemic index of food by acarbose and Plantago psyllium mucilage. Arch Med Res 1998 Summer;29(2):137-41); Rodriguez-Moran M, Guerrero-Romero F, Lazcano-Burciaga G. Lipid- and glucose-lowering efficacy of Plantago Psyllium in type II diabetes. J Diabetes Complications 1998 September-October; 12(5): 273-8; Frati Munari A C, Benitez Pinto W, Raul Ariza Andraca C, Casarrubias M. Lowering glycemic index of food by acarbose and Plantago psyllium mucilage. Arch Med Res 1998 Summer; 29(2): 137-41; Jenkins D J. Effect of method of administration of psyllium on glycemic response and carbohydrate digestibility. J Am Coll Nutr 1991 August; 10(4): 364-71; Pastors J G, Blaisdell P W, Balm T K, Asplin C M, Pohl S L. Psyllium fiber reduces rise in postprandial glucose and insulin concentrations in patients with non-insulin-dependent diabetes. Am J Clin Nutr 1991 June; 53(6): 1431-5; Welsh J D, Manion C V, Griffiths W J, Bird P C. Effect of psyllium hydrophilic mucilloid on oral glucose tolerance and breath hydrogen in postgastrectomy patients. Dig Dis Sci 1982 January; 27(1): 7-12; Jarjis H A, Blackburn N A, Redfern J S, Read N W. The effect of ispaghula (Fybogel and Metamucil) and guar gum on glucose tolerance in man. Br J Nutr 1984 May; 51(3): 371-8; Frati-Munari A C, Flores-Garduno M A, Ariza-Andraca R, Islas-Andrade S, Chavez Negrete A. Effect of different doses of Plantago psyllium mucilage on the glucose tolerance test. Arch Invest Med (Mex) 1989 April-June; 20(2): 147-52; Cherbut C, Bruley des Varannes S, Schnee M, Rival M, Galmiche J P, Delort-Laval J. Involvement of small intestinal motility in blood glucose response to dietary fibre in man. Br J Nutr 1994 May; 71(5): 675-85).

Side effects, such as allergic skin and respiratory reactions to psyllium dust, have largely been limited to people working in factories manufacturing psyllium products (Ebo D G, Stevens W J. IgE-mediated food allergyextensive review of the literature. Acta Clin Belg 2001 July-August;56(4):234-47).

Certain medications may interact with psyllium. Addition of psyllium husk twice a day to the regimen of a woman treated with lithium was associated with decreased lithium blood levels and lithium levels increased after psyllium was stopped. (Perlman B B. Interaction between lithium salt and ispaghula husk. Lancet 1990; 335: 416). Interactions with other drugs have been reported but these are very few. Psyllium slightly increases absorption of ethinyloestradiol while slowing down the rate of absorption. (Garcia J J, Fernandez N, Diez M J, Sahagun A, Gonzalez A, Alonso M L, Prieto C, Calle A P, Sierra M. Influence of two dietary fibers in the oral bioavailability and other pharmacokinetic parameters of ethinyloestradiol. Contraception 2000 November; 62(5): 253-7). Psyllium does not affect absorption of levothyroxine. (Psyllium Chiu A C, Sherman S I. Effects of pharmacological fiber supplements on levothyroxine absorption. Thyroid 1998 August; 8(8): 667-71). Psyllium also has little effect on the absorption of calcium. (Heaney R P, Weaver C M. Effect of psyllium on absorption of co-ingested calcium. J Am Geriatr Soc 1995 March; 43(3): 261-3). However, psyllium can affect the absorption of iron. (Fernandez R, Phillips S F. Components of fiber bind iron in vitro. Am J Clin Nutr 1982 January; 35(1): 100-6.; Rossander L. Effect of dietary fiber on iron absorption in man. Scand J Gastroenterol Suppl 1987; 129: 68-72). Psyllium does not affect absorption of digoxin. (Nordstrom M, Melander A, Robertsson E, Steen B. Influence of wheat bran and of a bulk-forming ispaghula cathartic on the bioavailability of digoxin in geriatric in-patients. Drug Nutr Interact 1987; 5(2): 67-9). Psyllium reduces the absorption of riboflavin (Roe D A, Kalkwarf H, Stevens J. Effect of fiber supplements on the apparent absorption of pharmacological doses of riboflavin. J Am Diet Assoc 1988 February; 88(2): 211-3).

Leung A Y, Foster S. Encyclopedia of Common Natural Ingredients Used in Food, Drugs, and Cosmetics, 2d ed. New York: John Wiley & Sons, 1996, 4279.

Voderholzer W A, Schatke W, MÃ¼hldorfer B E, et al. Clinical response to dietary fiber treatment of chronic constipation. Am J Gastroenterol 1997;92:958.

Anderson J W, Allgood L D, Turner J, et al. Effects of psyllium on glucose and serum lipid response in men with type 2 diabetes and hypercholesterolemia. Am J Clin Nutr 1999;70:46673.

Oson B H, Anderson S M, Becker M P, et al. Psyllium-enriched cereals lower blood total cholesterol and LDL cholesterol, but not HDL cholesterol, in hypercholesterolemic adults: Results of a meta-analysis. J Nutr 1997;127:197380.

Davidson M H, Dugan L D, Burns J H, et al. A psyllium-enriched cereal for the treatment of hypercholesterolemia in children: A controlled, double-blind, crossover study. Am J Clin Nutr 1996;63:96102.

Anderson J W, Davidson M H, Blonde L, et al. Long-term cholesterol-lowering effects as an adjunct to diet therapy in the treatment of hypercholesterolemia. Am J Clin Nutr 2000;71:14338.

Florholmen J, Arvidsson-Lenner R, Jorde R, Burhol P G. The effect of Metamucil on postprandial blood glucose and plasma gastric inhibitory peptide in insulin-dependent diabetics. Acta Med Scand 1982;212:2379.

Rodriguez-Moran M, Guerrero-Romero F, Lazcano-Burciaga G. Lipid- and glucose-lowering efficacy of plantago psyllium in type II diabetes. J Diabetes Complications 1998; 12:2738.

Anderson J W, Allgood L D, Turner J, et al. Effects of psyllium on glucose and serum lipid response in men with type 2 diabetes and hypercholesterolemia. Am J Clin Nutr 1999;70:46673.

Fernandez-Banares F, Hinojosa J, Sanchez-Lombrana J L, et al. Randomized clinical trial of Plantago ovata seeds (dietary fiber) as compared with mesalamine in maintaining remission in ulcerative colitis. Am J Gastroenterol 1999;94:42733.

Blumenthal M, Busse W R, Goldberg A, et al. (eds). The Complete Commission E Monographs: Therapeutic Guide to Herbal Medicines. Boston, Mass.: Integrative Medicine Communications, 1998, 1902.

Foster S. Herbs for Your Health. Loveland, Colo.: Interweave Press, 1996, 745.

Blumenthal M, Busse W R, Goldberg A, et al. (eds). The Complete Commission E Monographs: Therapeutic Guide to Herbal Medicines. Boston, Mass.: Integrative Medicine Communications, 1998, 1902.

Î²-sitosterol The major pharmacological effects of Î²-sitosterol are anti-inflammation, antiulcer, promotion of injured tissue. Soybean extract containing a minimum of 40% Î²-sitosterol as used in this invention (Sigma Chemicals Catalog S5753), which also contains campesterol, dihydro-brassicacasterol prepared according to the method of N. Kozumi, et al., Chem. Pharm. Bull., 27: 38, 1979. The source of Î²-sitosterol however is not relevant. It could be obtained from natural sources or from synthetic sources. Î²-sitosterol (C₂₉H₅₀O, molecular weight 414.72) is a common sterol in plants. It is generally isolated from wheat germ, soybean or corn oil.

Sterols are important cyclized triterpenoids that perform many critical functions in cells. Phytosterols such as campesterol, stigmasterol and Î²-sitosterol in plants, ergosterol in fungi and cholesterol in animals are each primary components of the cellular and sub-cellular membranes in their respective cell types. The dietary source of phytosterols in comes from vegetables and plant oils. The estimated daily phytosterol content in the conventional western-type diet is approximately 250 mg in contrast to a vegetable diet, which would provide double that amount. Although having no nutritional value to humans, phytosterols have recently received a great deal of attention due to their possible anti-cancer properties and their ability to decrease cholesterol levels when fed to a number of mammalian species, including humans. Phytosterols aid in limiting cholesterol absorption, enhance biliary cholesterol excretion and shift cholesterol from atherosclerotic plaque. While many of the mechanisms of action remain unknown, the relationship between cholesterol and phytosterols is apparent. This is perhaps not surprising given that chemically, phytosterols closely resemble cholesterol in structure. The major phytosterols are Î²-sitosterol, campesterol and stigmasterol. Others include stigmastanol (Î²-sitostanol), sitostanol, desmosterol, chalinasterol, poriferasterol, clionasterol and brassicasterol. (Gould R. G., Jones R. J., LeRoyu G. V., Wissler R. W., Taylor C. B.; Absorbability of B-sitosterol in humans; Metabolism, (August) 1969; 18 (8): 652-662; Tabata T., Tanaka M., Lio T.; Hypocholesterolemic activity of phytosterol. II; Yakugaku Zasshi, 1980; 100 (5): 546-552. Hepistall R. H., Porter K. A.; The effect of Î²-sitosterol on cholesterol-induced atheroma in rabbits with high blood pressure; Br. J. Experimental Pathology, 1957; 38: 49-54.) Several novel applications of phytosterols including Î²-sitosterol have been reported. The U.S. Pat. No. 5,965,449 to Novak describes a method of assessing risk for cardiovascular disease and other disorders and phytosterol-based compositions useful in preventing and treating cardiovascular disease and other disorders. The level of serum campesterol and Î²-sitosterol are determined and their ratio is correlated with the risk of cardiovascular or a related disorder. The U.S. Pat. No. 5,523,087 to Shlyankevich is for a pharmaceutical composition for the treatment of diabetic male sexual dysfunction; it contains physosterogens, phosphatidyl choline, Î²-sitosterol, Damiana leaf extract and vitamins and minerals. The U.S. Pat. No. 5,486,510 to Bouic, et al., is for a mixture of 12-sitosterol glucoside and Î²-sitosterol is administered to persons for the modulation or control of immune responses. The U.S. Pat. No. 5,747,464 to See is for a composition for inhibiting absorption of fat and cholesterol from the gut and a method for making and using the composition. The composition comprises Î²-sitosterol bound irreversibly to pectin to form a Î²-sitosterol and pectin complex. The U.S. Pat. No. 5,118,671 to Bombardelli, et al., is for complexes formed between aescin, cholesterol or Î²-sitosterol and phospholipids and a method for producing an anti-inflammatory effect is also described.

Simethicone. Simethicone is detergent, which is also used as a nonprescription drug used for short-term relief of excess gas in the gastrointestinal (GI) tract. It is also used to relieve symptoms of infant colic. Simethicone is available as a nonprescription product alone and in combination with nonprescription antacids, for relief of stomach upset. In the present invention, simethicone is used to improve dispersion of product and also to alleviate any symptoms of flatulence.

### SUMMARY OF INVENTION

The inventor has now discovered that appreciating the multi-factorial genesis of cholesterol elevation and diabetes and affecting several phases of cholesterol production simultaneously with a composition of natural substances is a way to effectively control blood cholesterol and sugar levels. A method of altering the concentration of the cholesterol constituents in the blood of a human to reduce the risk of atherosclerosis and vascular disease is provided. Simultaneously, a decrease in blood sugar levels both in those who are diabetic and those who are not is also demonstrated. A composition comprising psyllium husk, guggul extract, guar gum, chromium and ¹²-sitosterol is administered to a human in an amount effective to reduce or control blood cholesterol, to increase the concentration of HDL-cholesterol and/or to decrease the concentration of LDL-cholesterol in the blood of the human.

In accordance with the present invention, methods and compositions are provided for use in treating atherosclerosis and its associated diseases including cardiovascular disorders, cerebrovascular disorders, peripheral vascular disorders, intestinal vascular disorders and hyperglycemia. The methods and compositions of the present invention are particularly advantageous in that they may be used to both significantly lower plasma cholesterol levels and substantially arrest, reverse and/or cure the arterial plaque deposition and degenerative vascular wall changes associated with atherosclerosis. Additionally, surprising finding in the use of this composition is a significant lowering in blood sugar levels within a few weeks of therapy.

The compositions of the present invention can be administered prophylactically, so as to inhibit atherogenesis or restenosis, or therapeutically after atherogenesis has been initiated. Thus, for example, a patient who is to undergo balloon angioplasty can have a regimen of the composition administered substantially prior to the balloon angioplasty, preferably at least about a week or substantially longer. Alternatively, in a patient where atherogenesis is suspected, the administration the composition can begin at any time. As a prophylactic or treatment fir atherosclerotic susceptible hosts, the composition is chronically administered at an effective dosage. In one embodiment of the invention, the composition is administered to a human in one or more doses as a dietary supplement. In another embodiment of the invention, the composition is administered to a human in a pharmaceutical composition. In another aspect, the invention is a method of altering the concentration of cholesterol constituents in the blood of a human, to preferably reduce the risk of atherosclerosis and vascular disease, where the composition is administered to a human in an amount effective to increase the concentration of HDL-cholesterol in the blood of the human. Reducing cholesterol levels with the administration of this composition can also prevent other plaque formation and other types of atherosclerotic disease such as the cereberovascular complications of carotid artery plaques, peripheral vascular disease and claudication, and intestinal vascular blockage and infarction.

The composition of the present invention can be further administered prophylactically, so as to reduce the rise in blood sugar levels, either postprandial or upon fasting in both who are diabetic and those who are normoglycemic otherwise. It is an important and significant part of the discovery that the use of this invention can reduce the blood sugar levels in normoglycemic subjects. This is significant because of the established connection between sugar levels in the blood and heart disease.

### DETAILED DESCRIPTION

Hyperlipidemia relates to plasma cholesterol and triglyceride levels that exceed “normal”—arbitrarily defined as the 95^{th} percentile. But it is now clear that “ideal” or “optimal” levels are far, below the normal levels of the population. A large proportion of United States adults have concentrations above the optimal range and should be considered to have hyperlipoproteinemia. In a preferred embodiment of the invention, a composition is administered that simultaneously affects several different mechanisms in the production of atherosclerosis, including the levels of LDL and HDL cholesterol. In another embodiment, chromium is added for control of insulin and lipid metabolism and additional control or reduction of cholesterol levels. One theory is that both the presence of elevated plasma LDL and its oxidative modification within the artery wall is required to produce atherosclerosis. Indeed, then the use of an appropriate antioxidant in vivo should decrease the rate at which LDL is taken up by macrophage foam cells and slow the development of the fatty streak lesion. Chromium supplementation is also useful to treat elevated triglycerides. In a prospective, double-cross-over study of 14 men and 16 women supplementation with chromium picolinate for 2 months resulted in a statistically significant reduction in triglyceride levels of 17.4% (133 vs. 161 mg/dl; P<0.05).

Any dosage form may be employed for providing the patient with an effective dosage of the composition. Dosage forms include tablets, capsules, dispersions, suspensions, solutions, capsules, etc Pharmaceutically acceptable carriers include binding agents such as pregelatinized maize starch, polyvinylpryrrolidone or hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Tablets or capsules can be coated by methods well known to those of ordinary skill in the art. According to one aspect of the invention a composition is provided comprising a pharmaceutically acceptable combination of the composition and at least one carrier. Pharmaceutically acceptable carriers for inclusion into the present compositions include carriers most suitable for combination with insoluble drugs such as diluents, excipients and the like which enhance its oral administration. Suitable carriers include, but are not limited to, sugars, starches, cellulose and derivatives thereof, wetting agents, lubricants such as sodium lauryl sulfate, stabilizers, tabletting agents, anti-oxidants, preservatives, coloring agents and flavoring agents. As will be appreciated, the pharmaceutical carriers used to prepare compositions in accordance with the present invention will depend on the administrable form to be used. According to one embodiment of the invention, the novel composition of the present invention comprises psyllium husk powder, Î²-sitosterol as 40% soybean extract, guggul gum extract, and guar gum, chromium chelated with amino acid along with simethicone and other flavoring and sweetening and binding agents to formulate a powder form of the preparation. Oral dosage forms formulated in accordance with standard pharmaceutical practice may be employed The administration of the composition is preferably in accordance with a predetermined regimen, which may be at least once daily and over an extended period of time as a chronic treatment, and could last for one year or more, including the life of the host. The dosage administered will depend upon administration frequency, the blood level desired, other concurrent therapeutic treatments, the condition's severity, whether the treatment is for prophylaxis or therapy, the patient's age, the severity of cholesterol elevation, and the like. In a preferred aspect of the invention, a composition of the present invention is administered to reduce or control blood cholesterol levels in persons having a total cholesterol of 240 mg/DL (5.95 mmMl/L) or higher. In another embodiment of the invention, the compositions are administered to reduce levels of LDL-cholesterol in persons with an LDL-cholesterol of 130 mg/dL (3.41 mmMl/L) or higher. In yet another embodiment of the invention, the compositions are administered to reduce triglycerides in persons having blood triglycerides of 200 mg/dL (2.26 mmMl/L) or higher. In another embodiment, a composition of the present invention is administered to raise levels of HDL to persons with an HDL-cholesterol of 35 mg/dL (1.04 mmoM/L) or lower to reduce the risk of atherosclerosis associated with low HDL levels. In yet another embodiment of the invention, the compositions are administered to reduce blood sugar levels in both hyperglycemic and normoglycemic patients. The compositions and methods of the present invention may also be utilized to improve or maintain vascular health in specific organ systems including the cardiovascular system, the cereberovascular system, the peripheral vascular system and the intestinal vascular system. The composition and methods of the present invention may also be utilized to reduced blood sugar levels. According to an additional embodiment, the compositions of the present invention may be admixed with food.

### EXAMPLE

Using conventional techniques, the following formulation (9.36 g/sachet) was manufactured by Innovative Health Products (Largo, Fla.):

[Composition of Invention; 9.35 g]

| | |
|---|---|
| Psyllium Husk Powder | 6 g |
| Simeticone 30% | 0.133 g |
| Silica | 0.0571 g |
| BÎ²sitosterol | 0.32 g |
| Gum Guggul Extract | 0.22 g |
| Guar Gum | 0.30 g |
| Chromium | 200 mcg (as 2 mg Chromium |
| | Chelate) |
| Orange Supreme Flavor | 1.60 g |
| Acesulfate Potassium | 9.38 g |
| Powder | |
| Citric Acid | 0.35 g |

Twenty six subjects having total plasma cholesterol of between 240 and 300 mg/dL were selected for inclusion in the statistical study. All subjects received 9.34 G of the product composition twice a day 15 minutes before meal with 16 ounces of water for 20-days when their basal values such as blood glucose on fasting, totally weight, percent fat lost, cholesterol and triglyceride levels were monitored. The tests were repeated at 30-days. On a statistical basis, the group lost 2.5% body weight within 20 days and 5.70% body weight in 30 days. The loss in body fat was remarkable at 7 and 10% respectively for those two evaluation periods. The reduction in fasting blood glucose was 5% and postprandial levels by 10% in 30 days and the loss of cholesterol were almost 8%. There was a 10% reduction in triglycerides in 30 days. These results were surprising even when considering the additive effects of the components. A consistent decrease in blood sugar concomitantly with decrease in blood cholesterol will have a significant effect on the cardiovascular disease status of a patient and furthermore prevent such illness when used prophylactically.

The invention has been described in detail with particular reference to preferred embodiment thereof. However, it will be appreciated that those skilled in the art, upon consideration of this disclosure may make variations and modifications within the spirit and scope of the invention.

1. A method of reducing or controlling blood cholesterol, triglycerides or sugar in a mammal comprising the administration to said mammal a composition of between 2 g and 10 g of psyllium husk, 0.05 g to 0.2 g of simethicone (30%), 0.1 g to 0.5 g of Î²-sitosterol, 0.1 g to 5 g of guggul tree (Commiphora mukul) extract, 0.1 g to 0.5 g of guar (Cyamopis tetragonoloba) gum, and 10 to 500 mcg of chromium.

2. The method of claim 1 wherein said composition further comprises a pharmaceutically acceptable carrier.

3. The method of claim 2 wherein said composition further comprises a flavoring agent, a sweetening agent.

4. The method of claim 1 wherein the chromium is chromium picolinate, or chromium tripicolinate or a complex of chromium with an amino acid.

5. The method of claim 1 where in the Î²-sitosterol is an equivalent quantity of soybean extract containing not less than 40% Î²-sitosterol.

6. The method of claim 1 wherein said composition is administered 1-3 times per day.
